(19) United States
(12) Patent Application Publication
(10) Pub. No.: US 2025/0073050 A1
(43) Pub. Date: Mar. 6, 2025

(54) LOCKING ARTICULATED ARTIFICIAL FINGER ASSEMBLY

(71) Applicant: Daniel Didrick, Mead, CO (US)

(72) Inventor: Daniel Didrick, Mead, CO (US)

(21) Appl. No.: 18/460,645

(22) Filed: Sep. 4, 2023

(51) Int. Cl. A61F 2/58; A61F 2/80

(52) U.S. Cl. CPC A61F 2/586; A61F 2/80; A61F 2002/6854

(57) ABSTRACT

An artificial finger assembly includes a residual finger socket adapted to receive a residual finger of the wearer, and a hinge arranged rearwardly of the residual finger socket. The assembly further includes a pair of controlling arms pivotally coupled to the hinge and including a first controlling arm and a second controlling arm coupled to the residual finger socket. A middle finger portion extends forwardly of the socket and is configured to pivot relative to the residual finger socket. The first controlling arm is pivotally coupled to the middle finger portion. A fingertip portion is pivotally coupled to the middle finger portion. A lock assembly is configured to lock the pair of controlling arms together to prevent the pivoting of the controlling arms, the middle finger portion, and the fingertip portion.

### TECHNICAL FIELD

The present disclosure relates, generally, to an artificial finger assembly. More particularly, the present disclosure pertains to an artificial finger assembly allowing an amputee to lock the assembly in addition to regaining control of the flexion and extension movements of an artificial finger.

### BACKGROUND INFORMATION

Presently the inner construction of an artificial finger is composed of a sponge-like filling. Occasionally, artificial fingers are created with fixable joints as used in action-figure leg joints. These joints provide for stationary positioning of the finger. Presently, technology with smart materials keeps many waiting for a new wave of prosthetic advancements. The problems faced with creating electronic artificial fingers for finger amputees are size and cost. Unfortunately, cost will prevent many in need from obtaining expensive electronic prostheses. Creating self-contained electronic finger prostheses offers additional difficulties due to the limited space provided. Attempts have been made to solve the problem of replacing a missing finger with a mechanical device that was more appropriately designed for a robot than a human.

### SUMMARY OF DISCLOSURE

One aspect of this disclosure relates to an artificial finger assembly adapted to be engaged with a residual finger of a wearer and configured to mimic flexion and extension of a finger. The artificial finger assembly includes a residual finger socket adapted to receive the residual finger of the wearer and adapted to engage with the residual finger. The finger assembly also includes a hinge arranged rearwardly of the residual finger socket, and a pair of controlling arms pivotally coupled to the hinge and including a first controlling arm and a second controlling arm coupled to the residual finger socket. The finger assembly further includes a middle finger portion extending forwardly of the residual finger socket and configured to pivot relative to the residual finger socket. The first controlling arm is pivotally coupled to the middle finger portion. Moreover, the finger assembly includes a fingertip portion arranged forwardly of the middle finger portion and pivotally coupled to the middle finger portion. Furthermore, the finger assembly includes a lock assembly configured to lock the pair of controlling arms together to prevent the pivoting of the controlling arms, the middle finger portion, and the fingertip portion in response to a downward force exerted by the residual finger on the residual finger socket when the fingertip portion is in contact with an object.

In some additional, alternative, or selectively cumulative embodiments, the hinge is a track hinge having an arcuate track and the second controlling arm is configured to slide along the arcuate track.

In some additional, alternative, or selectively cumulative embodiments, the first controlling arm is pivotally coupled to the hinge and the middle finger portion.

In some additional, alternative, or selectively cumulative embodiments, the residual finger socket includes a movable floor, and the lock assembly includes at least one locking gear coupled to the middle finger portion and the first controlling arm and including a plurality of teeth. The lock assembly also includes at least one locking arm pivotally coupled to the residual finger socket and including a hook. The hook engages with the plurality of teeth in response to pivoting of at least one locking arm due to the downward force applied on the movable floor when the fingertip portion is arranged contacting the object.

In some additional, alternative, or selectively cumulative embodiments, the fingertip portion includes a pair of fingertip cores pivotally coupled to the middle finger portion and defining a gap therebetween, a retractable pulley slidably arranged inside the gap and defining a channel, a spring arranged inside the channel, a spring controlling lever pivotally coupled to the pair of fingertip cores and arranged inside the channel and above the spring, and a toggle switch engaged with the spring controlling lever and configured to be displaced in a lateral direction to move the spring controlling lever between the first position and the second position. In the first position, the spring controlling lever is arranged inside a cutout of the retractable pulley to prevent sliding of the retractable pulley. In the second position, the spring controlling lever is arranged outside the cutout facilitating a compression of the spring in response to sliding of the retractable pulley due to the pivoting of the fingertip portion in a rearward direction when the fingertip portion abuts the object.

In some additional, alternative, or selectively cumulative embodiments, the residual finger socket is a ring. The lock assembly includes a block coupled to the second controlling arm and arranged above the ring, a pair of vertically extending pillars arranged spaced apart from each other, wherein lower ends of the pillars fixedly coupled to the ring, wherein the pillars extend inside the block and configured to slide in a vertical direction inside the block, a pair of spring arranged surrounding the pair of pillars and biasing the pillars to an upward position. The upper ends of the pillars are arranged contacting the pair of blocks in the upward position. The lock assembly also includes a first lever extending inside the block in a lateral direction and pivotally coupled to a bolt slidably coupling one of the pair of pillars to the block. The first lever pivots in response to the sliding of the associated pillar in the vertical direction relative to the block. The lock assembly further includes a second lever pivotally coupled to the first lever and extending in the lateral direction and configured to be removably engaged with the first controlling arm to lock the first and second controlling arms.

In some additional, alternative, or selectively cumulative embodiments, the second lever includes a protrusion and the first controlling arm includes at least one groove. The protrusion extends inside the groove in response to a downward movement of the pillar relative to the block when the ring is depressed downwardly upon engagement of the fingertip portion with the object.

In some additional, alternative, or selectively cumulative embodiments, the second lever moves towards the first controlling arm when the ring is depressed downwardly.

In some additional, alternative, or selectively cumulative embodiments, the second lever is disengaged from the first controlling arm upon removal of the downward force on the ring as the pillar moves upwardly due to a force applied by the springs.

Additional aspects and advantages will be apparent from the following detailed description of example embodiments, which proceeds with reference to the accompanying drawings.

### BRIEF DESCRIPTION OF THE DRAWINGS

FIG. 1 illustrates a perspective view of a finger prosthesis from which one strap is removed, in accordance with an embodiment of the disclosure;

FIG. 2 illustrates a perspective view of a portion of the finger prosthesis of FIG. 1, in accordance with an embodiment of the disclosure;

FIG. 3 illustrates an exploded view of the finger prosthesis of FIG. 1 with some of the component removed, in accordance with an embodiment of the disclosure;

FIG. 4 illustrates a bottom perspective view of a portion of the finger prosthesis with some of the components removed, in accordance with an embodiment of the disclosure;

FIG. 5 illustrates a top perspective view of the portion of finger prosthesis of FIG. 4 with a grip cap removed to show various components of a fingertip portion of the finger prosthesis, in accordance with an alternative embodiment of the disclosure;

FIG. 6 illustrates a rear perspective view of the portion of finger prosthesis of FIG. 5, in accordance with an alternative embodiment of the disclosure;

FIG. 7 illustrates a bottom perspective view of the portion of finger prosthesis of FIG. 5 with a lock shroud removed, in accordance with an alternative embodiment of the disclosure;

FIG. 8 illustrates a top perspective view of a finger prosthesis, in accordance with an embodiment of the disclosure;

FIG. 9 illustrates a rear perspective view of the finger prosthesis of FIG. 8 with a hand stabilizer bracket removed, in accordance with an embodiment of the disclosure;

FIG. 10 illustrates a rear top perspective view of the finger prosthesis of FIG. 9 with a rear block of a lock assembly removed, in accordance with an embodiment of the disclosure;

FIG. 11 illustrates a partial exploded view of the finger prosthesis of FIG. 9, in accordance with an embodiment of the disclosure; and

FIG. 12 illustrates a top perspective view of the portion of finger prosthesis of FIG. 9 with a rear block, a front block, and a lower controlling arm removed, in accordance with an alternative embodiment of the disclosure.

### DETAILED DESCRIPTION

Example embodiments are described below with reference to the accompanying drawings. Unless otherwise expressly stated in the drawings, the sizes, positions, etc., of components, features, elements, etc., as well as any distances therebetween, are not necessarily to scale, and may be disproportionate and/or exaggerated for clarity.

The terminology used herein is for the purpose of describing particular example embodiments only and is not intended to be limiting. As used herein, the singular forms “a,” “an” and “the” are intended to include the plural forms as well, unless the context clearly indicates otherwise. It should be recognized that the terms “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise specified, a range of values, when recited, includes both the upper and lower limits of the range, as well as any sub-ranges therebetween. Unless indicated otherwise, terms such as “first,” “second,” etc., are only used to distinguish one element from another. For example, one element could be termed a “first element” and similarly, another element could be termed a “second element,” or vice versa. The section headings used herein are for organizational purposes only and are not to be construed as limiting the subject matter described.

Unless indicated otherwise, the terms “about,” “thereabout,” “substantially,” etc. mean that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art.

Spatially relative terms, such as “right,” left,” “below,” “beneath,” “lower,” “above,” and “upper,” and the like, may be used herein for ease of description to describe one element's or feature's relationship to another element or feature, as illustrated in the drawings. It should be recognized that the spatially relative terms are intended to encompass different orientations in addition to the orientation depicted in the figures. For example, if an object in the figures is turned over, elements described as “below” or “beneath” other elements or features would then be oriented “above” the other elements or features. Thus, the term “below” can, for example, encompass both an orientation of above and below. An object may be otherwise oriented (e.g., rotated 90 degrees or at other orientations) and the spatially relative descriptors used herein may be interpreted accordingly.

Unless clearly indicated otherwise, all connections and all operative connections may be direct or indirect. Similarly, unless clearly indicated otherwise, all connections and all operative connections may be rigid or non-rigid.

Like numbers refer to like elements throughout. Thus, the same or similar numbers may be described with reference to other drawings even if they are neither mentioned nor described in the corresponding drawing. Also, even elements that are not denoted by reference numbers may be described with reference to other drawings.

Many different forms and embodiments are possible without deviating from the spirit and teachings of this disclosure and so this disclosure should not be construed as limited to the example embodiments set forth herein. Rather, these example embodiments are provided so that this disclosure will be thorough and complete, and will convey the scope of the disclosure to those skilled in the art.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments.

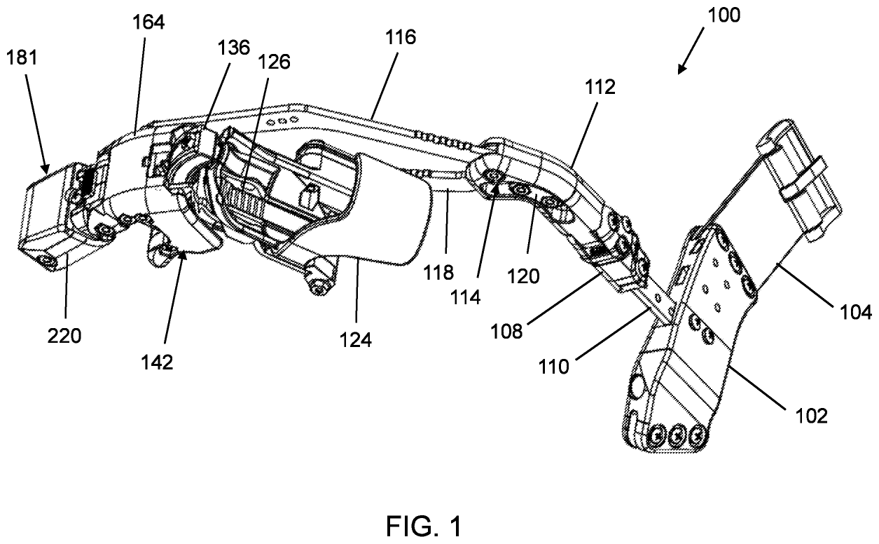

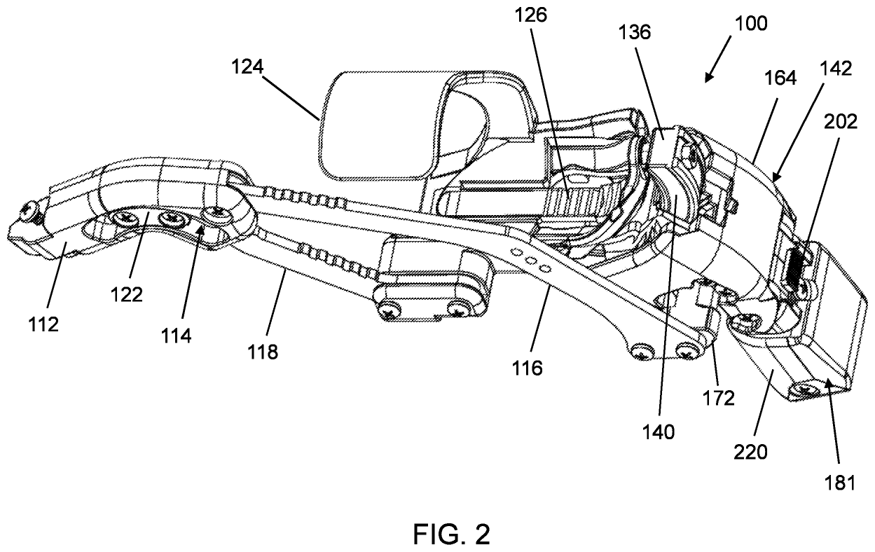

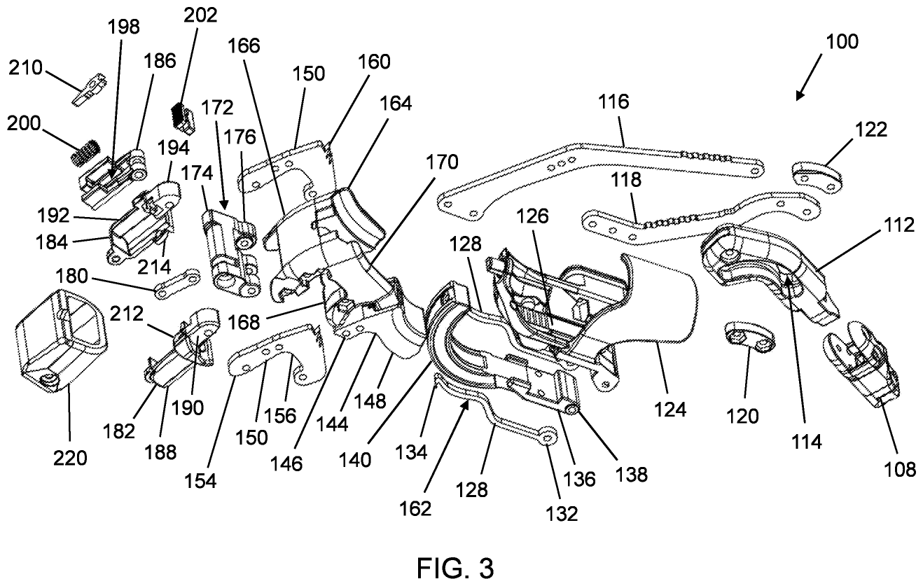

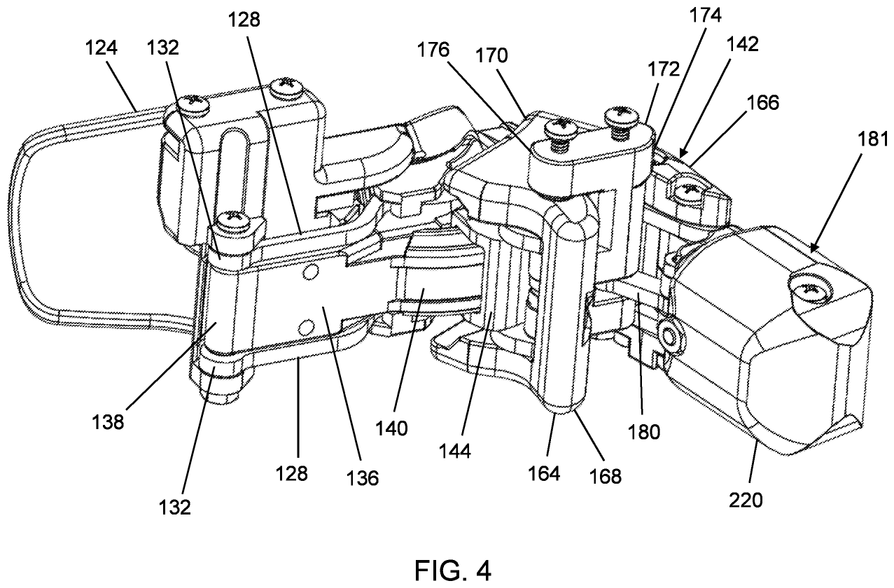

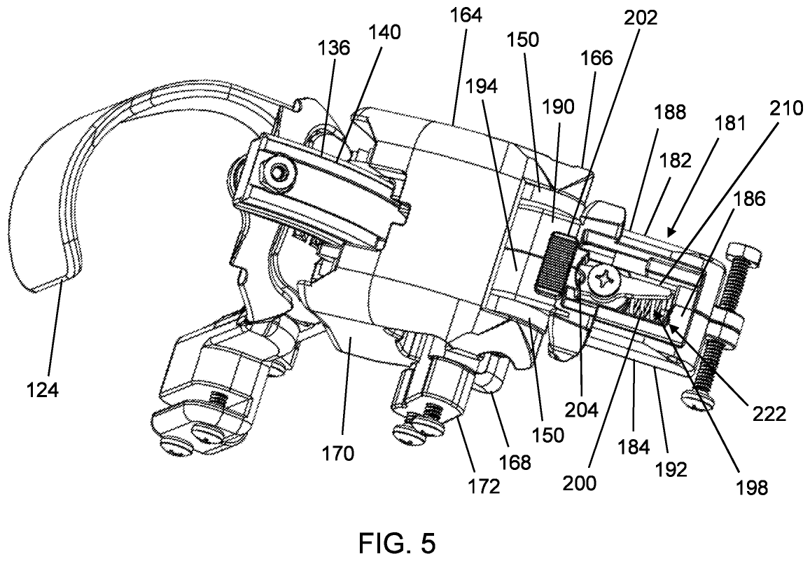

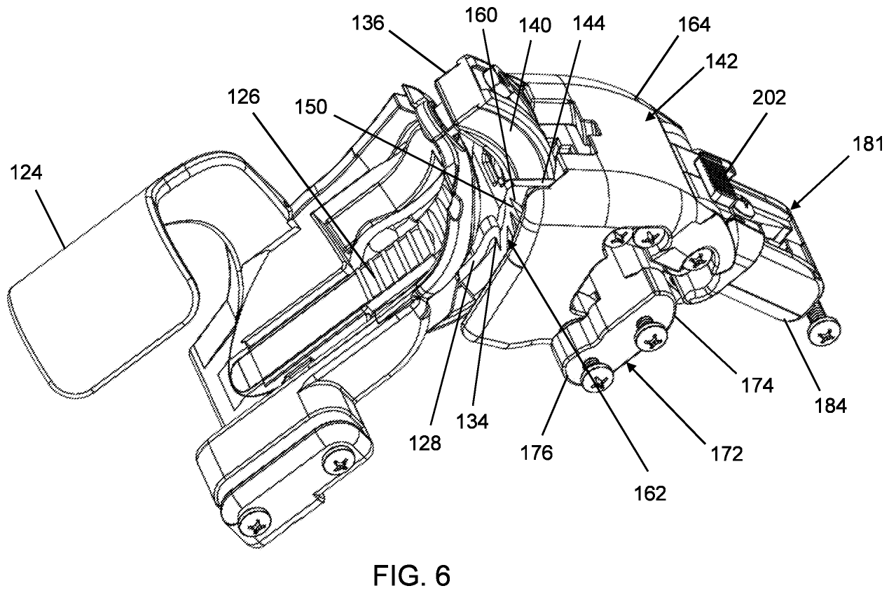

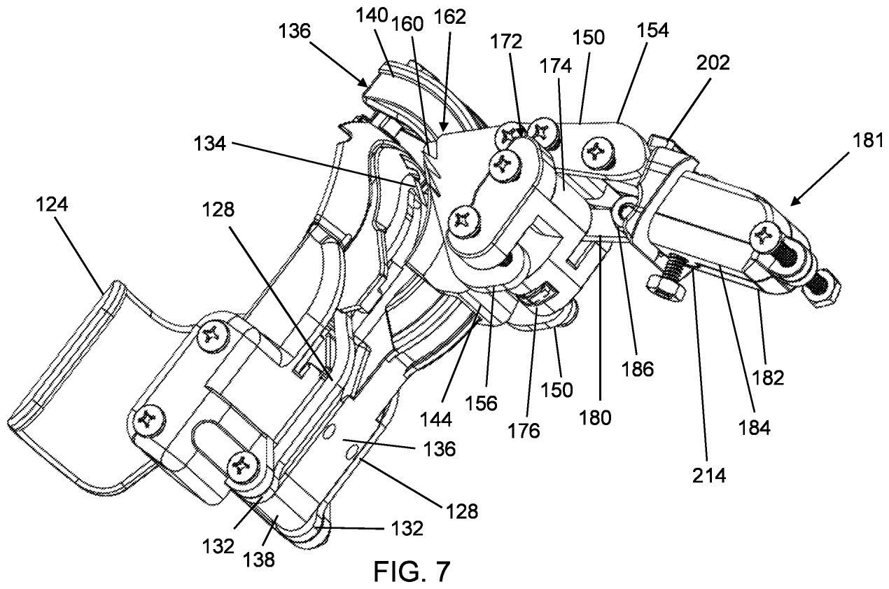

Referring to FIGS. 1 to 7, an artificial finger assembly 100, also referred to as a finger prosthesis 100, is shown, according to an example embodiment of the disclosure. The prosthesis 100 includes a series of reciprocally and transversely interconnected components that form the shape and replace the missing portion of an amputated finger. The movement of the artificial finger segments are controlled by the remaining portion of an amputated finger, also referred to as residual finger, when present, or an adjacent finger when a finger is completely missing using leverage.

As shown, the prosthesis 100 includes a hand stabilization bracket 102 adapted to be positioned on a palm of a wearer and a pair of bands 104 removably engaged with the hand stabilization bracket 102 and adapted to be fastened to the palm of the wearer. The bands 104 and the hand stabilization bracket 102 together provide a secure means of fastening each artificial finger desired by the user. The prosthesis 100 also includes a track hinge stabilization bracket 108 and a finger connection bracket 110 arranged between the track hinge stabilization bracket 108 and the hand stabilization bracket 102 and connecting the track hinge stabilization bracket 108 to the hand stabilization bracket 102.

Further, the prosthesis 100 includes a track hinge 112 having a first end connected to the hinge stabilization bracket 108 and a second end arranged distally from the hinge stabilization bracket 112. The track hinge 112 includes an arcuate channel 114 or groove i.e., track 114 extending between the first end and the second end in a longitudinal direction. Additionally, the prosthesis 100 includes a pair of controlling arms, for example, an upper controlling arm 116 (i.e., first controlling arm 116) and a lower controlling arm 118 (i.e., a second controlling arm 118), engaged to the track hinge 112 and configured to control the pivoting of various components of the prosthesis 100 to enable flexion and extension of the artificial finger assembly. A first end of the upper controlling arm 116 is pivotally engaged to the second end of the track hinge 112 using a single screw and is able to pivot between an extended position and a downward position without conflicting with the lower controlling arm 118.

As shown, the lower controlling arm 118 includes a curved shape and is slidably coupled with the tack hinge 112 and configured to slide along the track 114 and follow a path defined by the track 114. To enable the slidable engagement of the lower controlling arm 118 with the track hinge 112, the prosthesis 100 includes a pair of slidable track guides 120, 122 that are arranged on either side of the lower controlling arm 118 and hold the lower controlling arm 118 in position and prevents a lateral movement of the lower controlling arm 118 relative to the track hinge 112. As shown, the track guides 120, 122 are arranged/disposed at a first end of the lower controlling arm 118. The controlled movement of the lower controlling arm 118 along the curvature of the track 114 forces the lower controlling arm 118 to pivot in a manner that mimics the movement of a pivot point which is arranged at the center of the oval arc located beneath the track hinge 112. The movement of the curved lower controlling arm 118 mimics a pivot point of the user's metacarpal joint allowing an external mechanical device to follow the same bending pattern as the users controlling residual finger.

A second end of the lower controlling arm 118 is connected to a residual finger socket 124 of the prosthesis using two screws and nuts. The residual finger socket 124 is configured to receive the residual finger of the wearer and is engaged with the residual finger of the wearer. As shown, the residual finger socket 124 includes a first end to which the second end of the lower controlling arm 118 is attached, and a second end arranged distally to the lower controlling arm 118. Moreover, the socket 124 includes a moveable floor 126 that can be pressed down by the users' residual finger once the prosthesis 100 is pressed against an object allowing pressure to be exerted on it.

Further, the prosthesis includes at least one locking arm 128, for example, a pair of locking arms 128, coupled to the socket 124 and extending along a length of the socket 124. Each of the locking arms 128 includes a first end having an eye portion 132 and a second end having a hook 134. The eye portions 132 of the locking arms 128 are arranged inside a fork portion of the residual finger socket 124 located underneath the floor 126 of the residual finger socket 124 and are connected to the fork portion of the socket 124. The second ends of the locking arms 128 extend outwardly of the second end of the socket 124 in the longitudinal direction. Moreover, the prosthesis 100 includes a slide bracket 136 having an eye portion 138 and a curved bracket portion 140 extending in a longitudinal direction from the eye portion 138. The eye portion 138 is arranged at the first end of the socket 124 and inside the fork portion of the socket 124. The eye portion 138 of the slide bracket 136 and the eye portions 132 of the locking arms 128 are arranged inside the fork portion of the socket 124 such that the eye portions 132 of the locking arms 128 are arranged on opposite side of the eye portion 138 of the slide bracket 136 and the eye portions 132 of the locking arms 128 are arranged between the eye portion 138 of the slide bracket 136 and the forks of the fork portion of the socket 124. The socket 124, the locking arms 128 and the slide bracket 136 are coupled to each other via a single screw. Further, the curved bracket portion 140 of the slide bracket 136 is engaged with the second end of the socket 124 via a screw that extends in the longitudinal direction. As shown, a portion of the curved bracket portion 140 is arranged outwardly of the second end of socket 124 in the longitudinal direction.

Moreover, the prosthesis 100 includes a middle finger portion 142 having a sliding stabilizer 144 configured with the slide bracket 136 and includes a vertical extending channel from a bottom end to a top end, and the curved bracket portion 140 of the slide bracket 136 extends through the vertical channel. As can be seen, a free end of the curved bracket portion 140 extends outwardly of the top end of the sliding stabilizer 144 and is attached to the second end of the socket 124 via the screw. The sliding stabilizer 144 includes an inverted L shape such that a horizontal arm 146 extends outwardly in the longitudinal direction from the vertical channel. The sliding stabilizer 144 is arranged to slide up and down the slide bracket 136, simulating a pivot point at the center of the oval formed by both components when connected.

Additionally, prosthesis 100 includes at least one locking gear, for example, a pair of locking gears 150, arranged outwardly and on opposite sides of the sliding stabilizer 144 and coupled to the sliding stabilizer 144. Each of the locking gears 150 includes a substantially inverted L shape having a first portion 154 extending horizontally and in the longitudinal direction and a second portion 156 extending vertically downwardly of the first portion 154. In an assembly of the locking gears 150, the first portion 154 of each of the locking gears 150 extends along the horizontal arm 146 of the sliding stabilizer 144 and is connected to the horizontal arm 146 via two screws. Moreover, the second portions 156 of the locking gears 150 extend along a vertical arm 148 of the sliding stabilizer 144. Further, each of the locking gears 150 includes a plurality of teeth 160 extending rearwardly of the vertical arm 148 and are adapted to removably engage with the hooks 134 of the locking arms 128 to lock the relative pivoting of the various portions of the prosthesis 100. The locking arms 128 and the locking gears 150 together define a lock assembly 162 of the prosthesis 100.

To cover the sliding stabilizer 144 and the locking gears 150, the prosthesis 100 includes a lock shroud 164 having a horizontally oriented U shape and defining a cavity to receive the sliding stabilizer 144 and the locking gears 150. The lock shroud 164 includes an upper horizontally oriented arm portion 166, a lower horizontally oriented arm portion 168 arranged below and at a distance from the upper arm portion 166, and a vertically extending column portion 170 extending between the upper arm portion 166 and the lower arm portion 168. In the assembly of the locking gears 150 and the sliding stabilizer 144 with the lock shroud 164, the first portions 154 of the locking gears 150 and the horizontal arm 146 of the sliding stabilizer 144 are arranged inside the upper arm portion 166, while the second portions 156 of the locking gears 150 and the vertical arm 148 of the sliding stabilizer 144 are arranged inside the column portion 170. The upper arm portion 166, the first portions 154 of the locking gears 150 and the horizontal arm 146 are connected to each other via two screws. Further, the first portions 154 of the locking gears 150 extend forwardly of the horizontal arm 146 of the sliding stabilizer 144 in the longitudinal direction is coupled with an end of the upper arm portion 166 via single screw.

Further, the prosthesis 100 includes a lever 172 coupled to the shroud 164, the locking gears 150, and the upper controlling arm 116. As shown, the lever 172 includes a body 174 and a fork structure 176 extending rearwardly of the body 174. Fork structure 176 includes three fork arms, for example, a first fork arm, a second fork arm, and a third fork arm arranged spaced apart from each other and defining two cavities between them. For example, a first cavity is defined between the first fork arm and the second form arm, while a second cavity is defined between the second fork arm and the third fork arm. As shown, the first fork arm and the second fork arm extend inside the lower arm portion 168 of the shroud 164 and is coupled with the lower arm portion 168 via screws. Also, the second portion 156 of the one of the locking gears 150 is arranged between the first fork and the lower arm portion 168 and are coupled to each other via a first screw. Moreover, in the engagement of the lever 172 with the shroud 164, the third fork arm is arranged outside the shroud 172 such that a wall of the lower arm portion 168 of the shroud 164 and the other of the locking gears 150 is arranged between the third fork and the second fork. Further, the upper controlling arm 116 is connected to the third fork, the second fork, the locking gear 150, and the wall of the lower arm portion 168 via a second screw. The upper controlling arm 116 is connected with the shroud 164 and the lever 172 such that the third fork is arranged between the upper controlling arm 116 and a wall of the lower arm portion 168 in a lateral direction.

Additionally, the lever 172 is engaged to the shroud 164 such that the body 174 is arranged between the upper arm portion 166 and the lower arm portion 168 and extends forwardly and upwardly relative to the fork structure 176. The body 174 is coupled to the upper controlling arm 116 and a fingertip pulley 180 of the prosthesis 100 arranged inside a keyway of the body 174 via another screw. The fingertip pulley 180 is arranged proximate to the first fork in the lateral direction and extends outwardly and forwardly in the longitudinal direction from the shroud 164.

Furthermore, the prosthesis 100 includes a fingertip portion 181 having a first fingertip core 182, a second fingertip core 184, and a retractable pully 186 arranged between the first fingertip core 182 and the second fingertip core 184. The first fingertip core 182 includes a first front portion 188 and a first rear portion 190 arranged offset from each other in the lateral direction. Similarly, the second fingertip core 184 includes a second front portion 192 and a second rear portion 194 arranged offset from each other in the lateral direction. In the assembly, the first rear portion 190 and the second rear portion 194 abuts each other and are disposed inside the upper arm portion 168 of the shroud 164 and between the front portions of the locking gears 150 and are engaged with each other via a screw. Accordingly, the upper arm portion 168, the locking gears 150, and the rear portions 190, 194 of the fingertip cores 182, 184 are attached to each other via a screw. Therefore, the fingertip cores 182, 84 are pivotally coupled to the shroud 172 and the locking gears 150.

Further, the retractable pulley 186 slidably extends inside a gap defined between the first front portion 188 of the first fingertip core 182 and the second front portion 192 of the second fingertip core 184. As shown, the retractable pulley 186 includes a fork portion that is pivotally coupled to a front end of the fingertip pulley 180, and a housing portion arranged inside the gap and defining a channel 198. A spring 200 is arranged inside channel 198 and extends in the longitudinal direction. The retractable pulley 186 sits slidably engaged inside the fingertip cores 182, 184.

Moreover, the rear portions 190, 194 of the fingertip cores 182, 184 together define a keyway and a toggle switch 202 extends inside the keyway and is coupled to the fingertip cores 182, 184. The toggle switch 202 is adapted to slide between a first position and a second position in the lateral direction within the confines of the keyway. Also, the switch 202 includes a small nodule 204 that protrudes from the front of the switch 202 and fits inside of a groove disposed at a rear end of a spring controlling lever 210. The spring controlling lever 210 is arranged between the front portions 188, 192 of the fingertip cores 182, 184, above the spring 200 and is pivotably coupled with the fingertip cores 182, 184. As shown, the first fingertip core 182 includes a first bracket 212 arranged above the retractable pulley 186, while the second fingertip core 184 includes a second bracket 214 arranged downwardly of the retractable pulley 186, aligned with the first bracket 212. The spring controlling lever 210 is pivotally coupled with the first bracket 212 and the second bracket 214 and is adapted to pivot about central axes of the brackets 212, 214. The spring controlling lever 210 is adapted to pivot to a first position and a second position in response to the displacement of the toggle switch 202 to the first position and the second position, respectively.

Also, the prosthesis 100 includes a grip cap 220 configured to partially house the fingertip cores 182, 184 and is attached to the fingertip cores 182, 184 via a screw. Moreover, the screw engaging the spring controlling lever 219 with the first bracket 212 and the second bracket 214, extends through a lower wall of the grip cap 220 and attaches the spring controlling lever 210 with the grip cap 220. In the first position of the toggle switch 202, a tip of the spring controlling lever 210 is arranged inside a cutout 222 arranged at a forward end of the retractable pulley 186, while in the second position of the toggle switch 202, the tip of the spring controlling lever 210 is arranged outwardly of the cutout 222. Accordingly, in the first position, the fingertip portion 181 is restricted from pivoting rearwardly relative to a middle finger portion 142 when the grip cap 220 contacts an object. In the second position, the fingertip portion 181 is adapted to be pivoted rearwardly relative to the middle finger portion 142, enabling a movement of the retractable pulley 186 in a rearward direction. As the retractable pulley 186 moves rearwardly due to the rearward pivoting of the fingertip portion 181 upon a contact of the grip cap 220 with the object, spring 200 is compressed, enabling an improved grip of the prosthesis 100 on the object when holding the object.

The lock assembly 162 is configured to lock the finger assembly 100 and locks the pivoting of the controlling arms 116, 118, the middle finger portion 142, and the fingertip portion 181. The lock assembly 162 locks the finger assembly 100 when the hooks 134 of the locking arms 128 are in engagement with the teeth 160 of the locking gears 150. The locking arms 128 are engaged with the locking gears 150 in response to the pivoting of the locking arms 128 due to a downward push force applied on the movable floor 126 of the socket 124 when the fingertip 181 (i.e., grip cap 220) is arranged contacting an object. The lock on the pivotal movement of the controlling arms 116, 118, the middle finger portion 142, and the fingertip portion 181 is removed upon removal of the push down force on the movable floor 126.

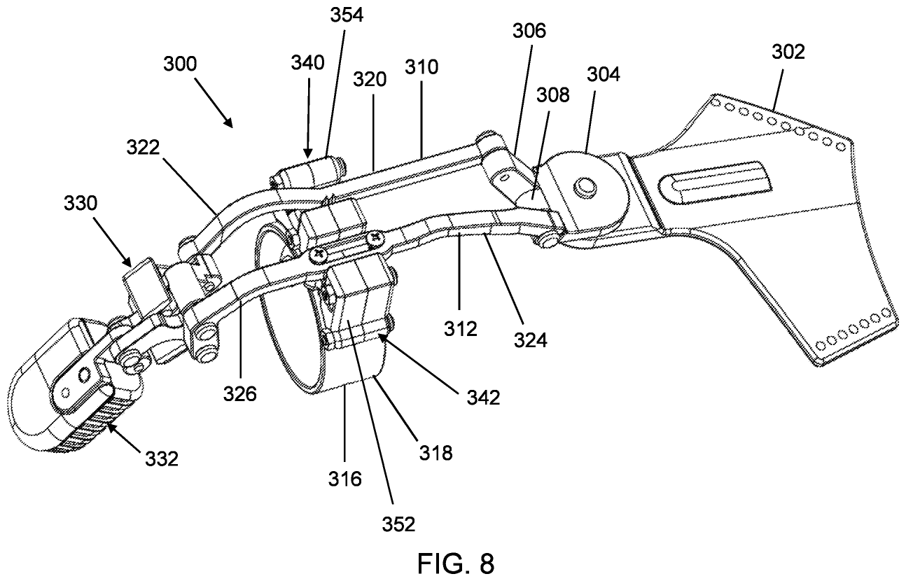

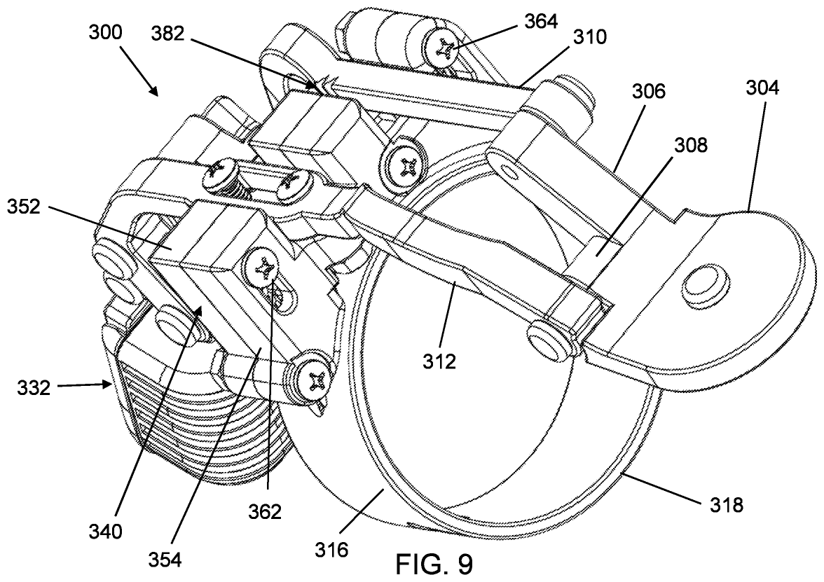

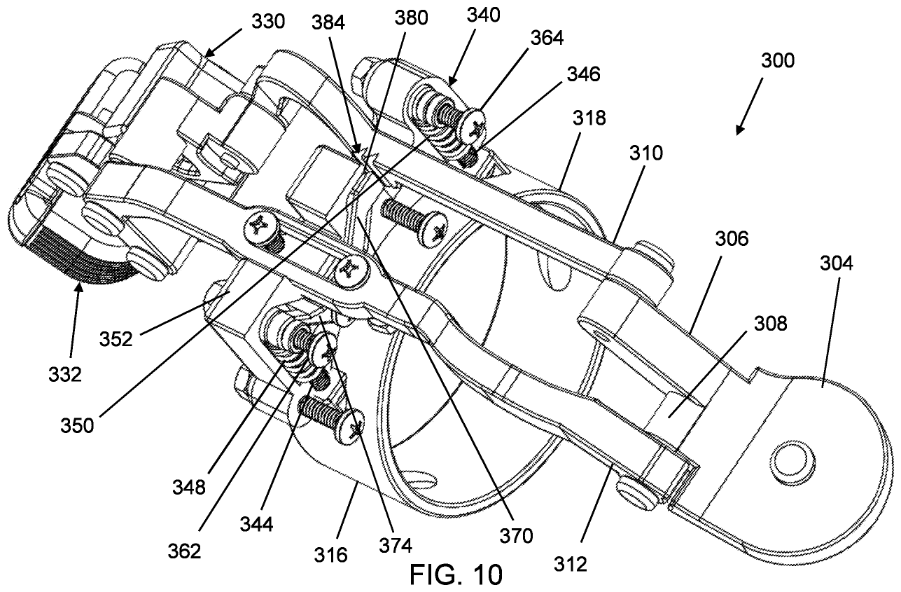

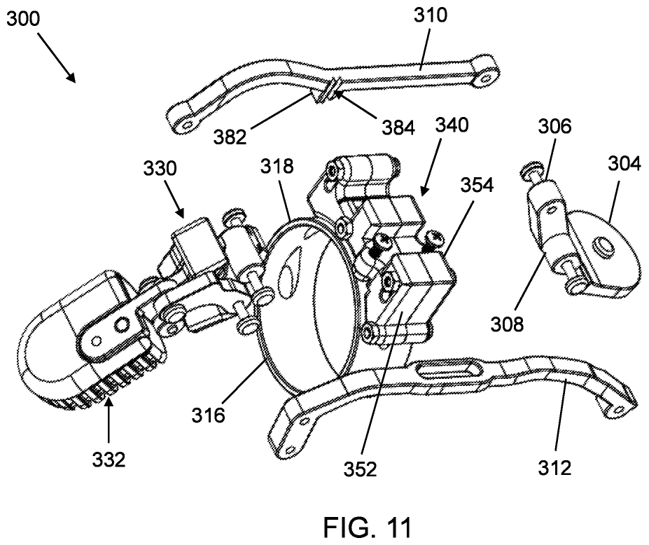

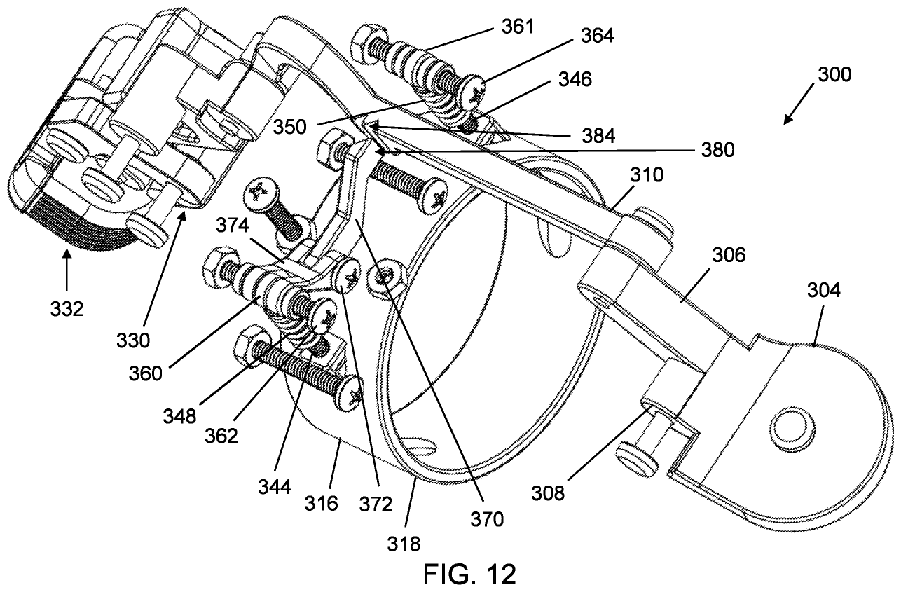

Referring to FIGS. 8 to 12, an artificial finger assembly 300 (i.e., prosthesis 300) is shown, according to an alternative embodiment of the disclosure. The prosthesis 300 includes a hand stabilization bracket 302 adapted to be engaged to a palm of the wearer, a hinge 304 coupled to the hand stabilization bracket 302 and having a pair of forks 306, 308 extending in a longitudinal direction and away from the hand stabilization bracket 302. Further, the prosthesis 300 includes a pair of controlling arms, for example, an upper controlling arm 310 (i.e., first controlling arm 310) and a lower controlling arm 312 (i.e., a second controlling arm 312), pivotally coupled to the pair of forks 306, 308. As shown, the upper controlling arm 310 is connected to an upper fork 306 of the pair of forks 306, 308, while the lower controlling arm 312 is connected to a lower fork 308 of the pair of forks 306, 308. Furthermore, the prosthesis 100 includes a residual finger socket 316, for example, a ring 318, arranged forwardly of the controlling arms 310, 312 and engaged to the lower controlling arm 312. The ring 318 is configured to receive a residual finger of the wearer and is supported on the residual finger of the wearer. As shown, the upper controlling arm 310 includes a first portion 320 that extends between the ring 318 and the hinge 304, and a second portion 322 that extends forwardly of the ring 318. Similarly, the lower controlling arm 312 includes a first portion 324 that is arranged between the hinge 304 and the ring 318 and a second portion 326 that extends forwardly in a longitudinal direction from the ring 318. As shown, the ring 318 and the controlling arms 310, 312 are arranged such that the ring 318 is arranged beneath the controlling arms 310, 312.

Moreover, the prosthesis 300 includes a middle finger portion 330 pivotally connected to the controlling arms 310, 312, and a fingertip portion 332 pivotally connected to the middle finger portion 330. The middle finger portion 330 and the fingertip portion 332 are configured to pivot in response to a pull-down force applied on the ring 318 by the wearer and hence on the controlling arms 310, 312 by the residual finger. Also, it may be appreciated that to enable pivoting of the middle finger portion 330 relative to the controlling arms 310, 312 and the fingertip portion 332 relative to the middle finger portion 330, the middle finger portion 330 includes a plurality of levers arranged and engaged with one or more of the controlling arms 310, 312 and the fingertip portion 332.

Further, the prosthesis 300 includes a lock assembly 340 to lock the pivoting of middle finger portion 330, the controlling arms 310, 312, and the fingertip portion 332. As shown, the lock assembly 340 includes a block 342, a pair of vertically extending pillars 344, 346, a pair of springs 348, 350, and a lever 370. The block 342 includes a front block 352 and a rear block 354 attached to each other via a plurality of screws and is arranged between the controlling arms 310, 312 and the ring 318 in the vertical direction. Further, the block 342 are attached to the lower controlling arm 312. Also, the pair of pillars 344, 346 are arranged spaced apart from each other and extends vertically upwardly from the ring 318 inside block 342. The pair of pillars 344, 346 are attached to ring 318 such that the ring 318 and pillars 344, 346 moves upwardly and downwardly together. Upper ends of the pillars 344, 346 include eye portions 360, 361 and horizontal bolts 362, 364 extend through the elongated sockets of the front block 352, the rear block 354, and the associated eye portions 360, 361 such that the eye portions 360, 361 are connected with the associated bolts 362, 364, Moreover, eye portions 360, 361 are arranged in contact the upper end of the block 342. Also, the pair of springs 348, 350 are arranged inside the block 342, surrounding the pillars 344, 346 such that upper ends the springs 348, 350 contact the associated eye portions 360, 361, while lower ends of the springs 348, 350 contact the block 342. Accordingly, as the ring 318 is pulled downwardly by the residual finger, the pillars 344, 346 moves vertically downwardly relative to the block 342, causing compression of the springs 348, 350. As the pull-down force is removed on the ring 318, the springs 348, 350 forces the pillars 344, 346 to move upwardly.

Further, the lock assembly 340 includes a first lever 374 and a second lever 370 arranged inside the block 342 and pivotally coupled teach other. The levers 374, 370 extend in a lateral direction between the pillars 344, 346. The second lever 370 includes a first end arranged proximate to the lower controlling arm 312 and pivotally coupled to a horizontally extending rod 372, and a second end arranged proximate to the upper controlling arm 310. Further, the second lever 370 includes a protrusion 380 arranged at the second end of the second lever 370 and configured to be removably engaged with the upper controlling arm 310 to lock the controlling arms 310, 312 with each other and hence lock the pivoting of middle finger portion 330, the fingertip portion 332, and the controlling arms 310, 312. To facilitate the engagement of the protrusion 380 with the upper controlling arm 310 and retain the protrusion 380 engaged with the upper controlling arm 310, the upper controlling arm 310 includes a tooth 382 that extends downwardly towards the block 342 from an elongated body of the upper controlling arm 310, and the tooth 382 includes at least one groove 384 arranged facing the second end of the lever 370 to receive the protrusion 380 for engaging the lever 370 with the upper controlling arm 310.

To pivot and move the second lever 370 towards the upper controlling arm 310, the first lever 374 is pivotally coupled to the bolt 362 that extends through the eye portion 362 of the pillar 344. As the ring 318 is pushed down by the residual finger, the pillars 344, 346 moves downwardly reactive to the block 342, causing a pivoting of the first lever 374 relative to the bolt 361 causing a movement of the second lever 370 towards the upper controlling arm 310. It may be appreciated that the upper controlling arm 310 is configured such that the upper controlling arm 310 moves back slightly during articulation in relation to the lower arm 312 and moves forward during extension. Accordingly, when the finger assembly 300 is articulated by pulling down the ring 318, the at least one groove 384 is arranged in alignment with the protrusion 380, with the protrusion 380 disposed away from the at least one groove 384. In the articulate position, when the fingertip portion contacts a surface and the ring 318 is depressed further, the second lever 370 moves towards the upper controlling arm 310 and the protrusion 318 extends inside the aligned groove 374 locking the finger assembly in the flexed/articulated position and prevents pivoting of the controlling arms 310, 312, the middle finger portion 330, and the fingertip portion 332.

To unlock the finger assembly, the downward force on the ring 318 is removed. This causes the pillars 344, 346 to move upwardly due to force applied by the springs 348, 350, causing the protrusion of the second lever 370 to move away from the groove 384, thereby disengaging the second lever 374 from the upper controlling arm 310, and enabling the pivoting of controlling arms 310, 312, the middle finger portion 330, and the fingertip portion 332.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims.

What is claimed is:

1. An artificial finger assembly adapted to be engaged with a residual finger of a wearer and configured to mimic flexion and extension of a finger, the artificial finger assembly comprising:
a residual finger socket adapted to receive the residual finger of the wearer and adapted to engage with the residual finger;
a hinge arranged rearwardly of the residual finger socket;
a pair of controlling arms pivotally coupled to the hinge and including a first controlling arm and a second controlling arm coupled to the residual finger socket;
a middle finger portion extending forwardly of the residual finger socket and configured to pivot relative to the residual finger socket, wherein the first controlling arm is pivotally coupled to the middle finger portion;
a fingertip portion arranged forwardly of the middle finger portion and pivotally coupled to the middle finger portion; and
a lock assembly configured to lock the pair of controlling arms together to prevent the pivoting of the controlling arms, the middle finger portion, and the fingertip portion in response to a downward force exerted by the residual finger on the residual finger socket when the fingertip portion is in contact with an object.

2. The artificial finger assembly of claim 1, wherein the hinge is a track hinge having an arcuate track and the second controlling arm is configured to slide along the arcuate track.

3. The artificial finger assembly of claim 2, wherein the first controlling arm is pivotally coupled to the hinge and the middle finger portion.

4. The artificial finger assembly of claim 1, wherein the residual finger socket includes a movable floor, and the lock assembly includes
at least one locking gear coupled to the middle finger portion and the first controlling arm and including a plurality of teeth, and
at least one locking arm pivotally coupled to the residual finger socket and including a hook, wherein
the hook engages with the plurality of teeth in response to pivoting of the at least one locking arm due to the downward force applied on the movable floor when the fingertip portion is arranged contacting the object.

5. The artificial finger assembly of claim 1, wherein the fingertip portion includes
a pair of fingertip cores pivotally coupled to the middle finger portion and defining a gap therebetween,
a retractable pulley slidably arranged inside the gap and defining a channel,
a spring arranged inside the channel,
a spring controlling lever pivotally coupled to the pair of fingertip cores and arranged inside the channel and above the spring, and
a toggle switch engaged with the spring controlling lever and configured to be displaced in a lateral direction to move the spring controlling lever between the first position and the second position, wherein
in the first position, the spring controlling lever is arranged inside a cutout of the retractable pulley to prevent a sliding of the retractable pulley, and
in the second position, the spring controlling lever is arranged outside the cutout facilitating a compression of the spring in response to sliding of the retractable pulley due to the pivoting of the fingertip portion in a rearward direction when the fingertip portion abuts the object.

6. The artificial finger assembly of claim 1, wherein the residual finger socket is a ring, and the lock assembly includes
a block coupled to the second controlling arm and arranged above the ring,
a pair of vertically extending pillars arranged spaced apart from each other, wherein lower ends of the pillars fixedly coupled to the ring, wherein the pillars extend inside the block and configured to slide in a vertical direction inside the block,
a pair of spring arranged surrounding the pair of pillars and biasing the pillars to an upward position, wherein upper ends of the pillars are arranged contacting the pair of blocks in the upward position,
a first lever extending inside the block in a lateral direction and pivotally coupled to a bolt slidably coupling one of the pair of pillars to the block, wherein the first lever pivots in response to the sliding of the associated pillar in the vertical direction relative to the block, and
a second lever pivotally coupled to the first lever and extending in the lateral direction and configured to be removably engaged with the first controlling arm to lock the first and second controlling arms.

7. The artificial finger assembly of claim 6, wherein the second lever includes a protrusion, and the first controlling arm includes at least one groove, wherein the protrusion extends inside the groove in response to a downward movement of the pillar relative to the block when the ring is depressed downwardly upon engagement of the fingertip portion with the object.

8. The artificial finger assembly of claim 7, wherein the second lever moves towards the first controlling arm when the ring is depressed downwardly.

9. The artificial finger assembly of claim 7, wherein the second lever is disengaged from the first controlling arm upon removal of the downward force on the ring as the pillar moves upwardly due to a force applied by the springs.
